(19) United States
(12) Patent Application Publication
(10) Pub. No.: US 2004/0097720 A1
(43) Pub. Date: May 20, 2004

(54) Use of glycodendrimer reagents for inhibiting adhesion by microorganisms

(76) Inventors: Richard R. Bott, Burlingame, CA (US); M. M. Cowan, Cincinnati, OH (US); Benjamin G. Davis, Durham (GB); John Bryan Jones, Lakefield (CA)

Correspondence Address: GENENCOR INTERNATIONAL, INC., 925 PAGE MILL ROAD, PALO ALTO, CA, 94304-1013, US

(21) Appl. No.: 10/417,768

(22) Filed: Apr. 16, 2003

Related U.S. Application Data
(63) Continuation-in-part of application No. 09/824,827, filed on Apr. 2, 2001, now Pat. No. 6,627,744.

(51) Int. Cl. C07H 21/04; C12N 9/64; C12N 1/21; C12P 21/02

(52) U.S. Cl. 536/23.2; 435/69.1; 435/226; 435/320.1; 435/252.3; 435/325

(57) ABSTRACT

The present invention is directed to glyodendrimeric proteases, composition with glycodendrimeric proteases and methods for inhibiting adhesion binding in microorganisms by contacting microorganisms with a glycodendrimeric protease or a composition with a glycodendrimeric protease. The invention may be used to treat patients in need of treatment.

### RELATED APPLICATION DATA

This application is a continuation-in-part of U.S. application Ser. No. 09/824,827 filed Apr. 2, 2001, which claims priority to U.S. application Ser. No. 09/347,029 filed on Jul. 2, 1999, U.S. Application Serial No. 60/131,446 filed on Apr. 28, 1999, U.S. Application Serial No. 60/091,687 filed on Jul. 2, 1998, which are hereby incorporated herein by reference in their entireties.

### FIELD OF THE INVENTION

The present invention is directed to glyodendrimeric proteases, compositions with glycodendrimeric proteases and methods for inhibiting adhesin binding in microorganisms by contacting microorganisms with a glycodendrimeric protease or a composition with a glycodendrimeric protease. The invention may be used to treat patients in need of treatment.

### BACKGROUND OF THE INVENTION

The emergence of drug-resistance among some pathogenic microorganisms necessitates a search for alternative methods to battle infections. Investigators have discovered and studied agents toxic to infecting microorganisms. Some investigators seek to fight disease by interrupting early stages of infection. Early stages include adhesion by a microorganism to a host tissue, which can be a prerequisite to establishing a harmful infection.

Adhesion can be mediated by microbial surface glycoproteins that act as markers in cell-cell communication events that determine microbial virulence (Sharon et al., Essays Biochem., 30:59-75 (1995)), inflammation (Lasky, Annu. Rev. Biochem., 64:113-139 (1995); Weis et al., Annu. Rev. Biochem., 65:441-473 (1996)), and host immune responses (Varki, Glycobiol., 3:97-130 (1993); Dwek, Chem. Rev., 96:683-720 (1996)). In addition, the correct glycosylation of proteins is critical to their expression and folding (Helenius, Mol. Biol. Cell, 5:253-265 (1994)) and increases their thermal and proteolytic stability (Opendakker et al., FASEB J., 7:1330-1337 (1993)). Glycoproteins occur naturally in a number of forms (glycoforms) (Rademacher et al., Annu. Rev. Biochem., 57:785-838 (1988)) that possess the same peptide backbone, but differ in both the nature and site of glycosylation.

Known strategies for reducing adhesion by a microorganism include using carbohydrate analogs to inhibit interactions between an adhesin molecule on a microorganism and a sugar on a host cell or tissue. (Zopf, D. et al., Adv. Exp. Med. Biol. 408:35-8 (1996); Ofek, I. and Doyle R. J., Bacterial Adhesion to Cells and Tissues; Chapman and Hall, New York (1994)). These analogs can be part of a carbohydrate cocktail, which includes carbohydrates having various structures corresponding to the binding specificities of one or more lectins of a microorganism. (Beuth, J. et al., Adv. Exp. Med. Biol. 408:51-56 (1996)). Manipulating gene regulation to prevent phenotype switching by the microorganism to an adhesion-plus variant provides yet another strategy for reducing adhesion by a microorganism. (Kahane, I. et al., Adv. Exp. Med. Biol. 408:107-111 (1996)).

For example, investigators have reported that, in the oral cavity, Streptococcus mutans attaches to glucans deposited on the tooth surface. (Koga, T. et al., J. Gen. Microbiol. 132:2873-2883 (1986)). Such attachment is believed to enhance the ability of S. mutans to metabolize dietary sucrose to acid, which then can destroy tooth enamel and eventually result in a carious lesion. S. mutans and other oral streptococci use a surface protein called glucan-binding lectin (GBL) to attach to surface-bound glucan. (Gibbons, R. J. et al., J. Bacteriol. 98:341-346 (1969)). Drake et al. developed an in vitro model system using soluble high-molecular weight glucans, also know as “dextrans,” and whole cell suspensions of S. cricetus to examine GBL binding. (Drake, D. et al., Infect. Immun. 56:1864-1872 (1988); Drake, D. et al., Infect. Immun. 56:2205-2207 (1988)). The glucan binding results in aggregation that is quantifiable with spectrophotometry.

WO 02/079394, which is hereby incorporated by reference, including any drawings, discloses the synthesis of chemically modified mutant protein, called also glycodendrimeric proteases; the subject matter of the application is also disclosed in US 02/10903, which is hereby incorporated by reference, including any drawings.

Given the prevalence of harm caused by infection with microorganisms, and the medical effort and cost devoted to fighting these infections, there is a need for additional and improved compositions and methods for fighting infection by microorganisms. There is also a need for additional and improved compositions and methods for reducing adhesion by microorganisms to and in animal tissues, and for reducing adhesion by microorganisms to dental prostheses.

### SUMMARY OF THE INVENTION

The present invention is directed to glyodendrimeric proteases, composition with glycodendrimeric proteases and methods for inhibiting adhesion of microorganisms by contacting microorganisms with a glycodendrimeric protease or a composition with a glycodendrimeric protease.

A first embodiment of the invention comprises a glyodendrimeric protease, the glycodendrimeric protease having a branched or multiply-branched structure that presents a carbohydrate moeity at each branch extremity wherein the glycodendrimeric protease inhibits adhesion of one or more microorganisms to a surface. The carbohydrate moeity is at least one selected from the group consisting of glucose, galactose, fucose, mannose, sialic acid, N-Acetylgalactosamine, N-Acetylglucosamine, and glucuronic acid. Other preferred carbohydrates can be found, for example, in WO 02/079394. The surface may be selected from the group consisting of other bacterial cells, human or animal cells, tissues, extracellular matrix, teeth and prostheses. The microorganism may be a prokaryote, a eukaryote, a virus or a combination thereof. The prokaryote may be a gram-positive bacterium, a gram-negative bacterium or a gram-variable bacterium. The prokaryote may be an Actinomyces, Staphylococcus or an E. coli. The eukaryote may be a fungus or protozoan. The fungus may be Candida. The glycodendrimeric protease may inhibit adhesion by binding to a microbial adhesin, preferably a lectin, and causing the degradation of the proteinaceous lectin, among other mechanisms.

A second embodiment of the invention is drawn to a composition comprising a glyodendrimeric protease, the glycodendrimeric protease having a branched or multiply-branched structure that presents a carbohydrate moeity at each branch extremity wherein the glycodendrimeric protease inhibits adhesion of one or more microorganisms to a surface. The carbohydrate moeity is at least one selected from the group consisting of glucose, galactose, fucose, mannose, sialic acid, N-Acetylgalactosamine, N-Acetylglucosamine, and glucuronic acid. Other preferred carbohydrates can be found, for example, in WO 02/079394. The surface may be selected from the group consisting of other bacterial cells, human or animal cells, tissues, extracellular matrix, teeth and prostheses. The microorganism may be a prokaryote, a eukaryote, a virus or a combination thereof. The prokaryote may be a gram-positive bacterium, a gram-negative bacterium or a gram-variable bacterium. The prokaryote may-be an Actinomyces, Staphylococcus or an E. coli. The eukaryote may be a fungus or protozoan. The fungus may be Candida. The glycodendrimeric protease may inhibit adhesion by binding to a microbial adhesin, preferably a lectin, and causing the degradation of the proteinaceous lectin, among other mechanisms.

A third embodiment of the invention provides a method for inhibiting adhesin binding in a microorganism comprising contacting the adhesin with a glycodendrimeric protease having a branched or multiply-branched structure that presents a carbohydrate moeity at each branch extremity wherein the glycodendrimeric protease inhibits adhesion of one or more microorganisms to a surface. The carbohydrate moeity is at least one selected from the group consisting of glucose, galactose, fucose, mannose, sialic acid, N-Acetylgalactosamine, N-Acetylglucosamine, and glucuronic acid. Other preferred carbohydrates can be found, for example, in WO 02/079394. The surface may be selected from the group consisting of other bacterial cells, human or animal cells, tissues, extracellular matrix, teeth and prostheses. The microorganism may be a prokaryote, a eukaryote, a virus or a combination thereof. The prokaryote may be a gram-positive bacterium, a gram-negative bacterium or a gram-variable bacterium. The prokaryote may be an Actinomyces, Staphylococcus or an E. coli. The eukaryote may be a fungus or protozoan. The fungus may be Candida. The glycodendrimeric protease may inhibit adhesion by binding to a microbial adhesin, preferably a lectin, and causing the degradation of the proteinaceous lectin, among other mechanisms.

A fourth embodiment of the invention provides a method of reducing binding of a microorganism to a surface comprising contacting the surface with a glycodendrimeric protease having a branched or multiply-branched structure that presents a carbohydrate moeity at each branch extremity wherein the glycodendrimeric protease inhibits adhesion of one or more microorganisms to a surface. The carbohydrate moeity is at least one selected from the group consisting of glucose, galactose, fucose, mannose, sialic acid, N-Acetylgalactosamine, N-Acetylglucosamine, and glucuronic acid. Other preferred carbohydrates can be found, for example, in WO 02/079394. The surface may be selected from the group consisting of other bacterial cells, human or animal cells, tissues, extracellular matrix, teeth and prostheses. The microorganism may be a prokaryote, a eukaryote, a virus or a combination thereof. The prokaryote may be a gram-positive bacterium, a gram-negative bacterium or a gram-variable bacterium. The prokaryote may be an Actinomyces, Staphylococcus or an E. coli. The eukaryote may be a fungus or protozoan. The fungus may be Candida. The glycodendrimeric protease may inhibit adhesion by binding to a microbial adhesin, preferably a lectin, and causing the degradation of the proteinaceous lectin, among other mechanisms.

A fifth embodiment of the invention provides a method of reducing adhesion by a microorganism to mammalian tissues or cells comprising contacting the mammalian tissues or cells with a glycodendrimeric protease having a branched or multiply-branched structure that presents a carbohydrate moeity at each branch extremity wherein the glycodendrimeric protease inhibits adhesion of one or more microorganisms to a surface. The carbohydrate mocity is at least one selected from the group consisting of glucose, galactose, fucose, mannose, sialic acid, N-Acetylgalactosamine, N-Acetylglucosamine, and glucuronic acid. Other preferred carbohydrates can be found, for example, in WO 02/079394. The surface may be selected from the group consisting of other bacterial cells, human or animal cells, tissues, extracellular matrix, teeth and prostheses. The microorganism may be a prokaryote, a eukaryote, a virus or a combination thereof. The prokaryote may be a gram-positive bacterium, a gram-negative bacterium or a gram-variable bacterium. The prokaryote may be an Actinomyces, Staphylococcus or an E. coli. The eukaryote may be a fungus or protozoan. The fungus may be Candida. The glycodendrimeric protease may inhibit adhesion by binding to a microbial adhesin, preferably a lectin, and causing the degradation of the proteinaceous lectin, among other mechanisms.

A sixth embodiment of the invention provides a method of treating a disease or disorder of a patient in need of such treatment comprising administering to the patient a glycodendrimeric protease having a branched or multiply-branched structure that presents a carbohydrate moeity at each branch extremity wherein the glycodendrimeric protease inhibits adhesion of one or more microorganisms to a surface. The carbohydrate moeity is at least one selected from the group consisting of The carbohydrate moeity is at least one selected from the group consisting of glucose, galactose, fucose, mannose, sialic acid, N-Acetylgalactosamine, N-Acetylglucosamine, and glucuronic acid. Other preferred carbohydrates can be found, for example, in WO 02/079394. The surface may be selected from the group consisting of other bacterial cells, human or animal cells, tissues, extracellular matrix, teeth and prostheses. The microorganism may be a prokaryote, a eukaryote, a virus or a combination thereof. The prokaryote may be a gram-positive bacterium, a gram-negative bacterium or a gram-variable bacterium. The prokaryote may be an Actinomyces, Staphylococcus or an E. coli. The eukaryote may be a fungus or protozoan. The fungus may be Candida. The glycodendrimeric protease may inhibit adhesion by binding to a microbial adhesin, preferably a lectin, and causing the degradation of the proteinaceous lectin, among other mechanisms. The patient may be an animal or a human. Administration can be accomplished by any method suitable for delivering the glycodendrimeric protease to the site of the disease or disorder. For example, administration of the enzyme to the animal or human can be oral or topical. Administration can optionally be targeted to mammalian tissues infected by tissue destroying pathogens, or to the nose, ear, vagina, skin, lungs or digestive tract of the mammal.

A seventh embodiment of the invention provides a method for reducing adhesion by a microorganism to mammalian oral tissues or cells or to a dental prosthesis comprising providing glycodendrimeric protease having a branched or multiply-branched structure that presents a carbohydrate moeity at each branch extremity wherein the glycodendrimeric protease inhibits adhesion of one or more microorganisms to a surface. The carbohydrate moeity is at least one selected from the group consisting of glucose, galactose, fucose, mannose, sialic acid, N-Acetylgalactosamine, N-Acetylglucosamine, and glucuronic acid. Other preferred carbohydrates can be found, for example, in WO 02/079394. The surface may be selected from the group consisting of other bacterial cells, human or animal cells, tissues, extracellular matrix, teeth and prostheses. The microorganism may be a prokaryote, a eukaryote, a virus or a combination thereof. The prokaryote may be a gram-positive bacterium, a gram-negative bacterium or a gram-variable bacterium. The prokaryote may be an Actinomyces, Staphylococcus or an E. coli. The eukaryote may be a fungus or protozoan. The fungus may be Candida. The glycodendrimeric protease may inhibit adhesion by binding to a microbial adhesin, preferably a lectin, and causing the degradation of the proteinaceous lectin, among other mechanisms. The patient may be an animal or a human. Administration can be accomplished by any method suitable for delivering the glycodendrimeric protease to the site of the disease or disorder. For example, administration of the enzyme to the animal or human can be oral or topical. Administration can optionally be targeted to mammalian tissues infected by tissue destroying pathogens, or to the nose, ear, vagina, skin, lungs or digestive tract of the mammal. The method may include administering to a mammal's oral cavity an oral care composition including an effective amount of the glycodendrimeric protease. Advantages of this method can include reducing adhesion by one or more microorganisms to teeth; reducing dental caries, plaque or calculus; reducing co-aggregation of microorganisms; reducing pellicle formation, inhibiting glucosyltransferase or a combination thereof.

An eighth embodiment of the invention provides an oral care composition comprising an effective amount of glycodendrimeric protease and optionally one or more other substances, the glycodendrimeric protease having a branched or multiply-branched structure that presents a carbohydrate moeity at each branch extremity wherein the glycodendrimeric protease inhibits adhesion of one or more microorganisms to a surface. The carbohydrate moeity is at least one selected from the group consisting of glucose, galactose, fucose, mannose, sialic acid, N-Acetylgalactosamine, N-Acetylglucosamine, and glucuronic acid. Other preferred carbohydrates can be found, for example, in WO 02/079394. The surface may be selected from the group consisting of other bacterial cells, human or animal cells, tissues, extracellular matrix, teeth and prostheses. The microorganism may be a prokaryote, a eukaryote, a virus or a combination thereof. The prokaryote may be a gram-positive bacterium, a gram-negative bacterium or a gram-variable bacterium. The prokaryote may be an Actinomyces, Staphylococcus or an E. coli. The eukaryote may be a fungus or protozoan. The fungus may be Candida. The glycodendrimeric protease may inhibit adhesion by binding to a microbial adhesin, preferably a lectin, and causing the degradation of the proteinaceous lectin, among other mechanisms. Oral care compositions of the invention include, but are not limited to, a mouthwash, a toothpaste, an implant or a combination thereof, and may optionally be in the form of a solid, a semi-solid, a liquid or an aerosol. Administration of the oral care composition to the mammal's oral cavity can be accomplished by any method suitable for delivering a composition to the oral cavity. For example, administration of the oral care composition can include rinsing with a liquid, applying a semisolid with a toothbrush, swab or syringe, implanting a solid or a combination thereof. Optionally, the oral care composition can be used to treat a dental prosthesis, either in the oral cavity or outside the oral cavity. Such a treatment can include applying to a dental prosthesis removed from a mammal's oral cavity an oral care composition including an effective amount of the composition.

### BRIEF DESCRIPTION OF THE DRAWINGS

FIG. 1 shows a shows a proposed mechanism of action for glycodendrimeric proteases of the present invention;

FIG. 2 shows a proposed synthesis for glycodendrimeric proteases. 1-4. MTS carbohydrate reagents 1-4 were used to modify the introduced cysteine thiol group (FIG. 1a). One mono-antennary carbohydrate structure 1 (21) and four multi-antennary

structures 2-4 were constructed. (35) To explore the effect of branching substructure

the

core branching structures of 2-4 were based upon both the conformationally flexible tris(2-aminoethyl)amine (TREN) core 6 to create 2b, 3, 4 and the rigid mesitylene core

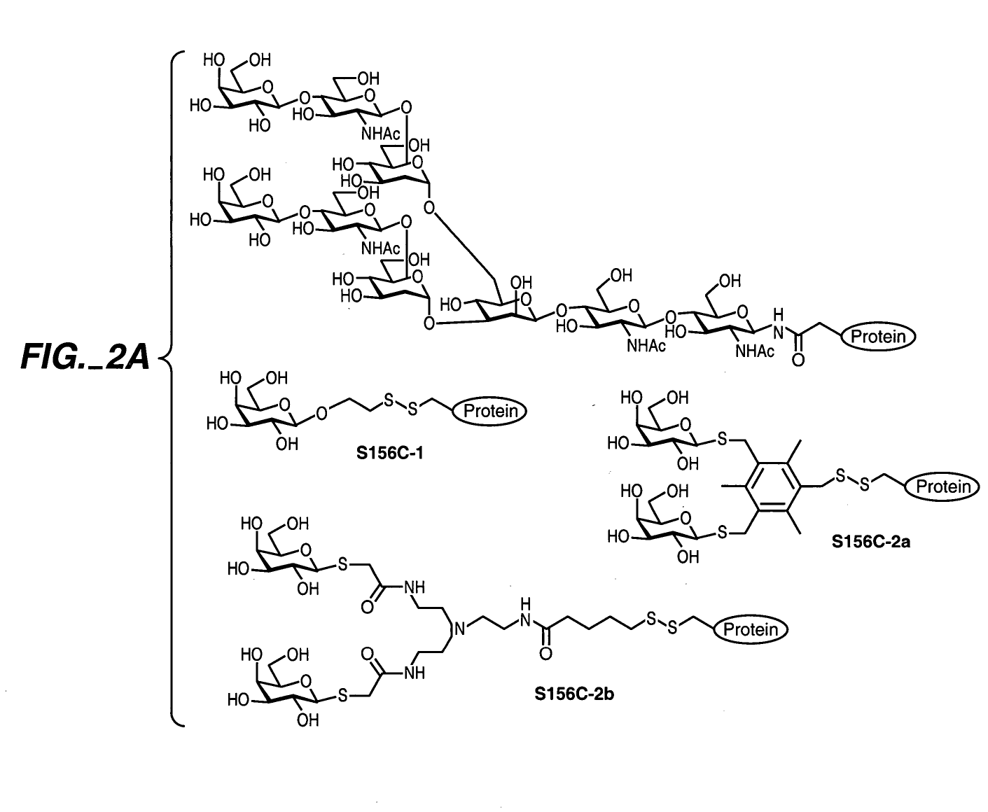

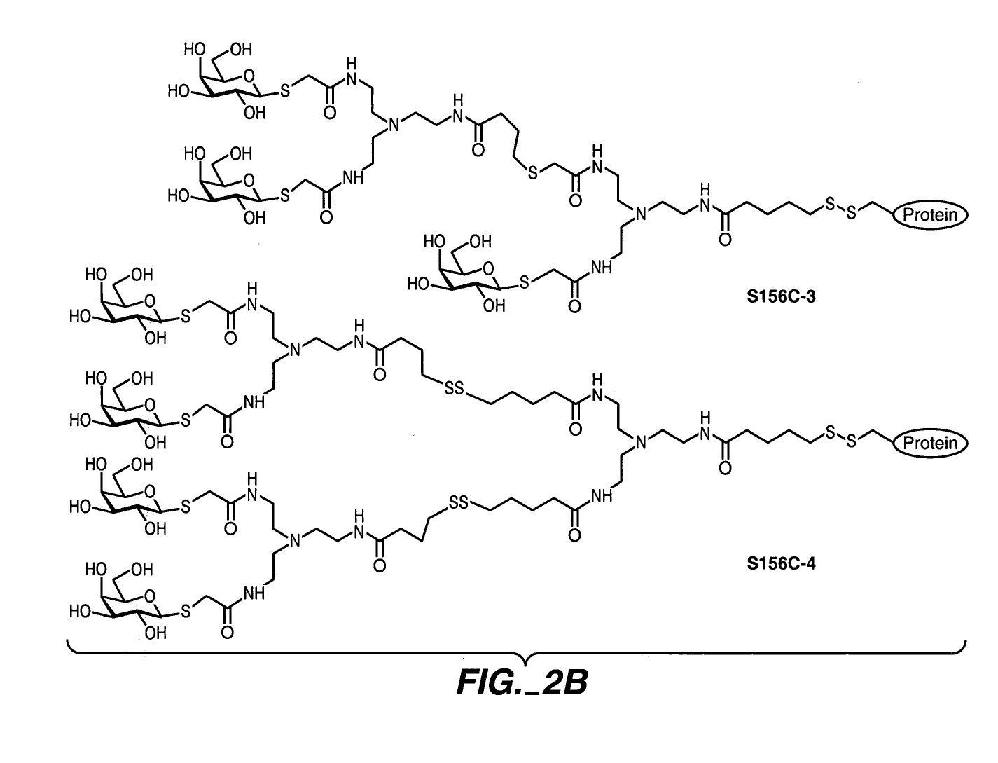

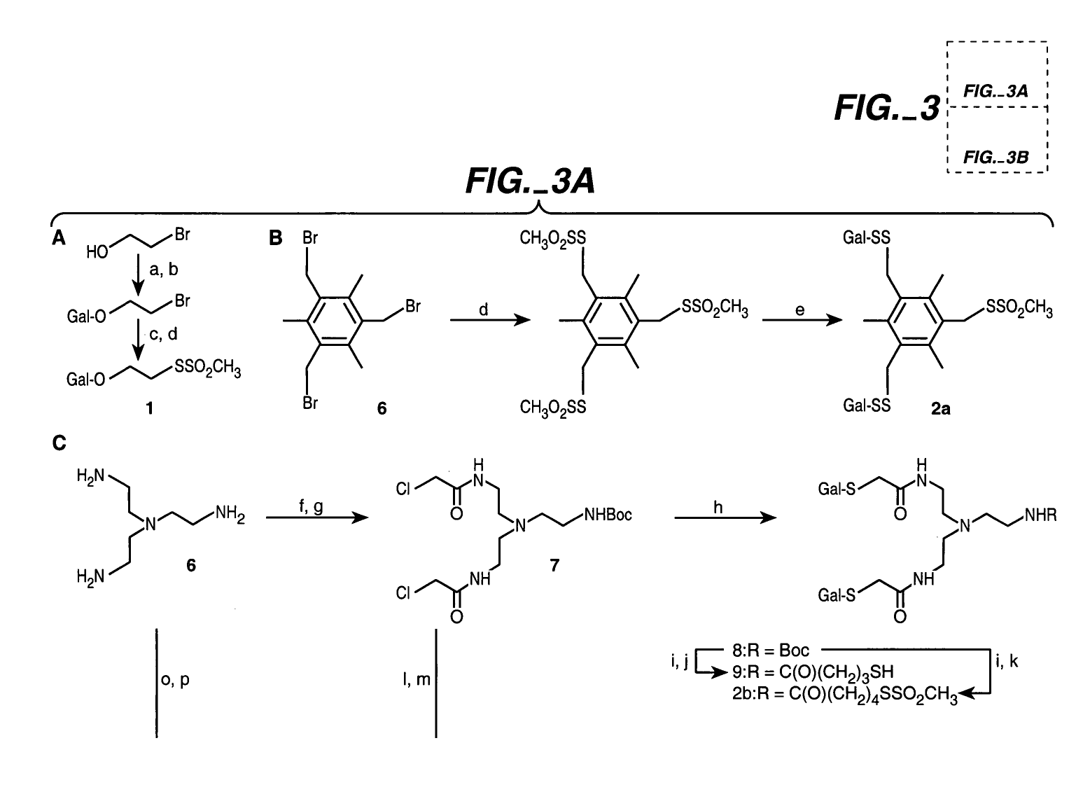

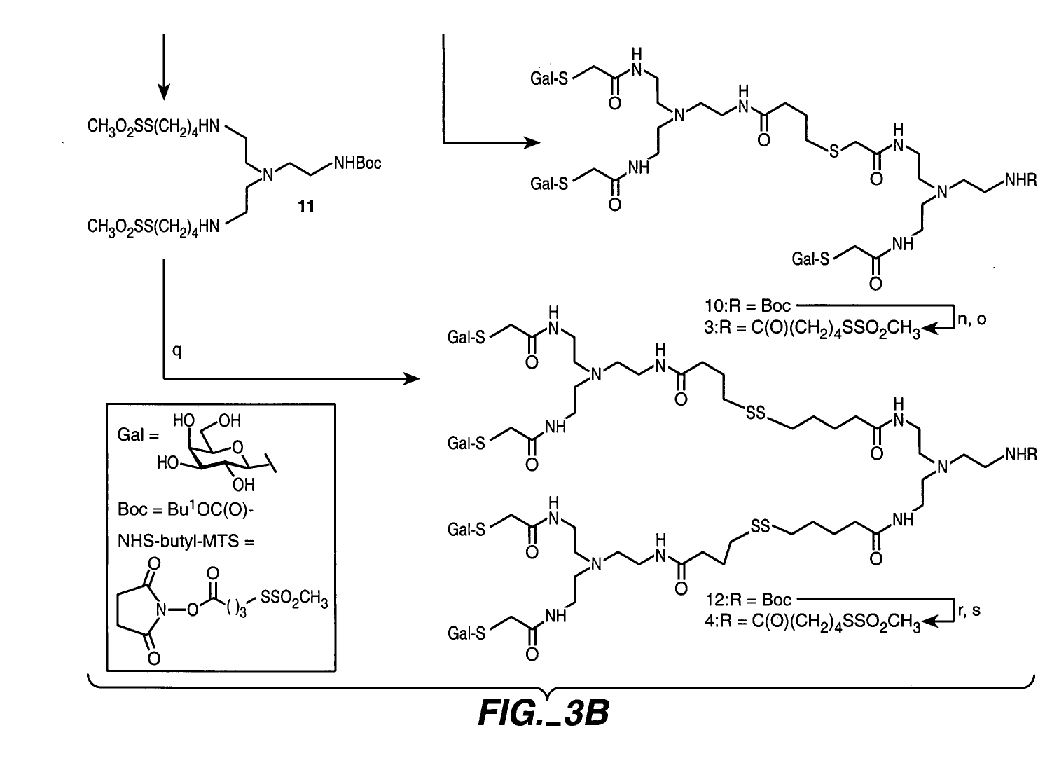

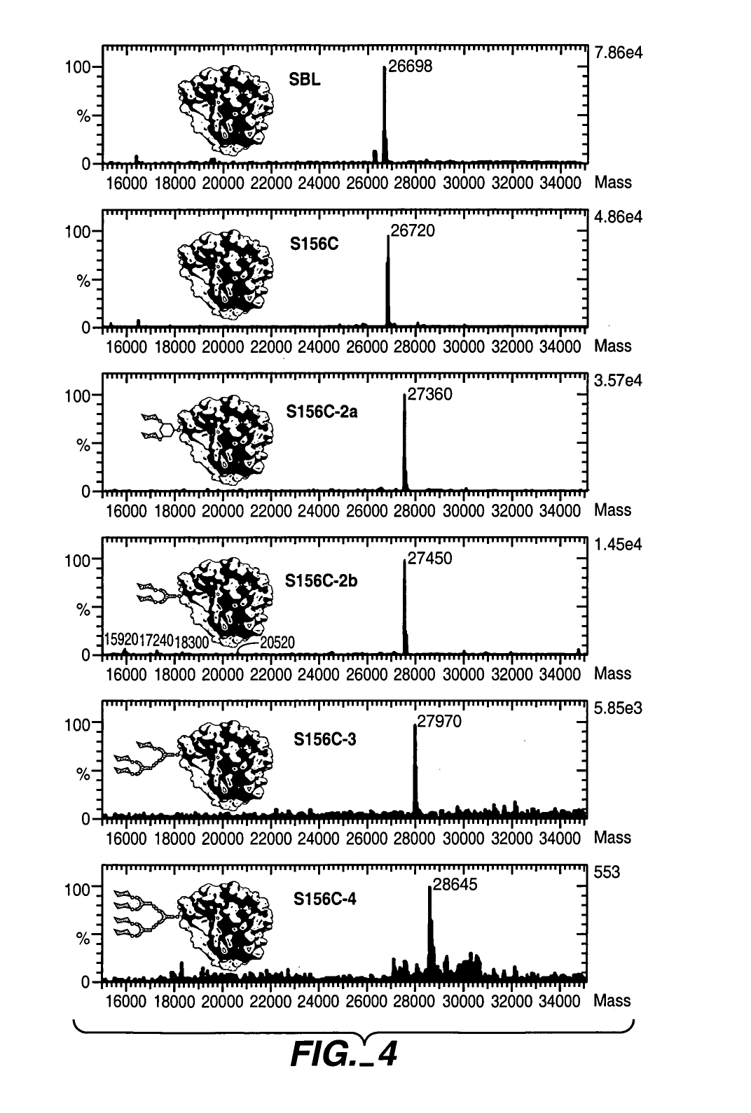

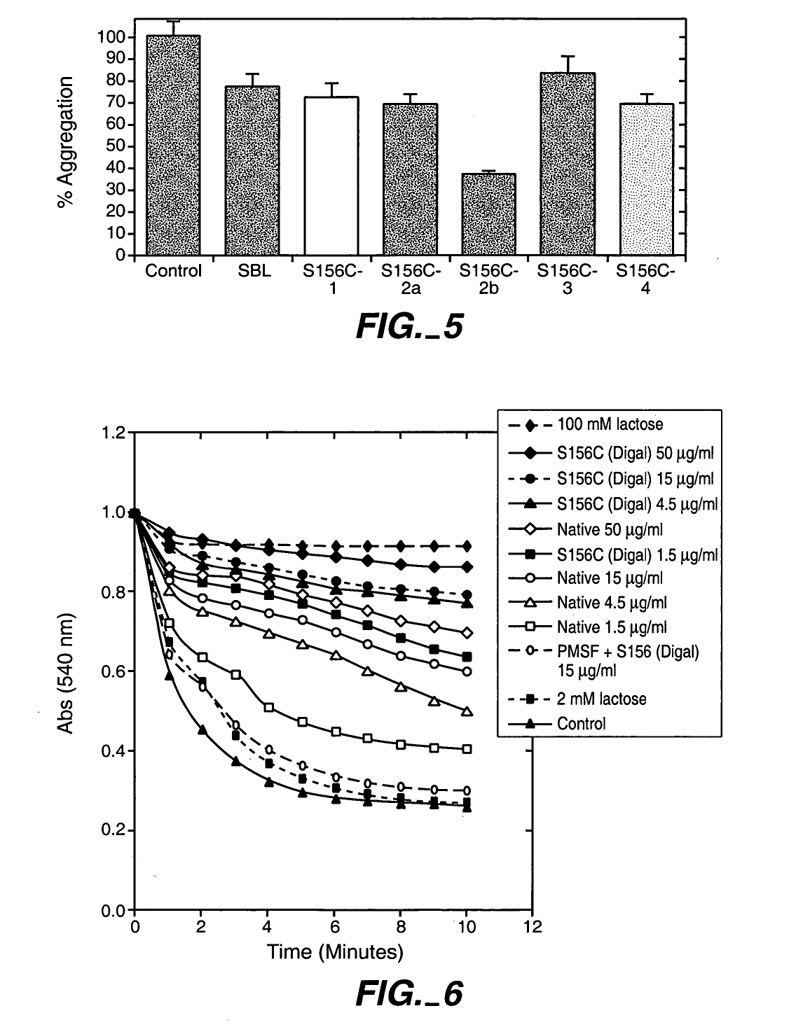

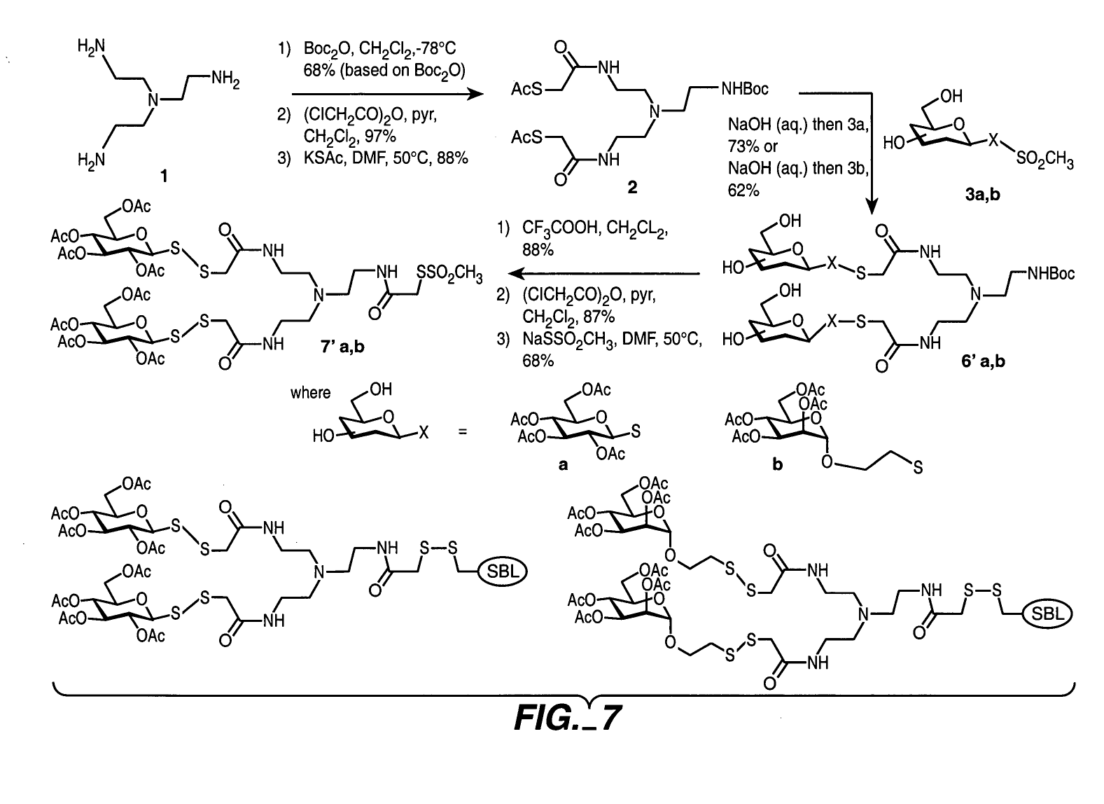

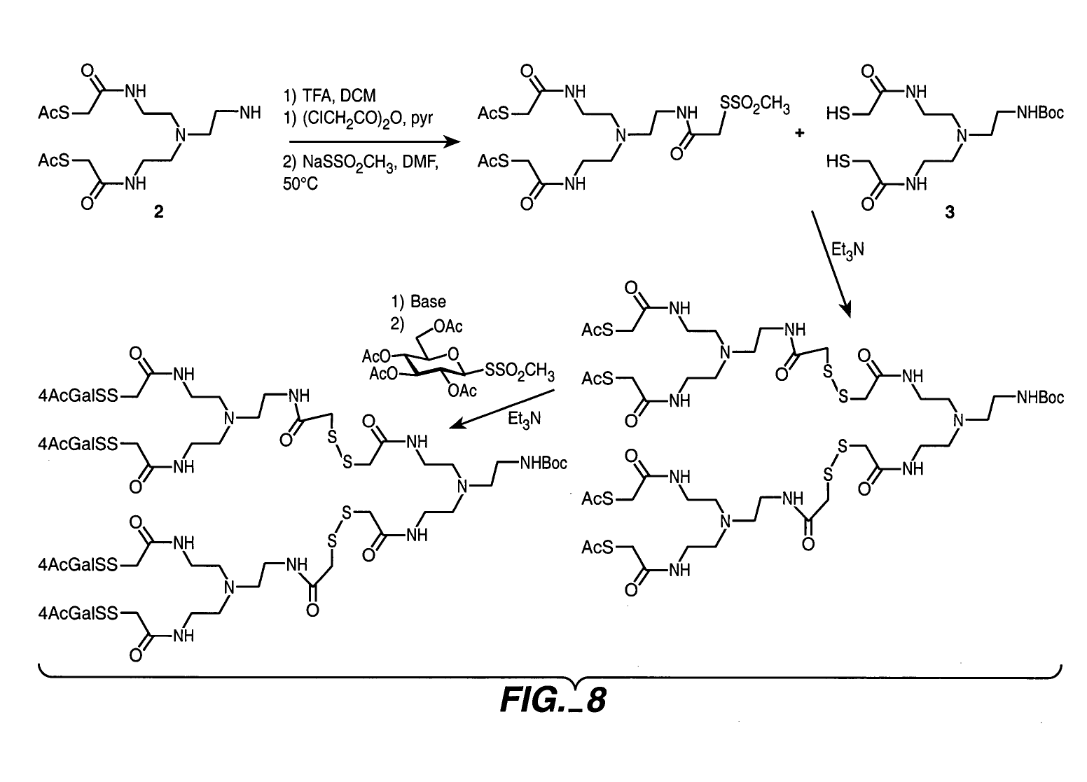

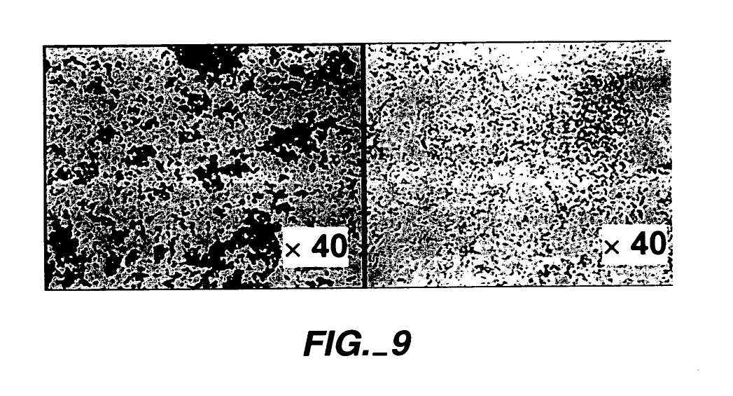

5 to create 2a (FIG. 2). Since our model target pathogen, A. naeslundii, utilizes galactose (Gal)-specific binding adhesins (36) to bind Gal-containing glycoproteins, (37)

Gal units were attached to the extremities of these dendrimer cores using 1-thio-®Dgalactopyranose (Gal-SH) as a reagent. Through appropriate sequential use of the branched building blocks 7, 9, and 11 and Gal-SH, the basic multiantennary structures 8, 10 and 12 were constructed. 8, 10 and 12 were then elaborated using the novel heterobifunctional linker reagent NHS-butyl-MTS to create a final amide bond using Nhydroxysuccinimide (NHS) activated esters to give glycodendrimer reagents 2b, 3 and

4. While reagents 2a, 2b and 4 are symmetrical, this methodology also allowed the synthesis of an asymmetric tri-antennary reagent 3, designed to mimic the asymmetric carbohydrate display that is observed in tri-antennary N-linked glycoproteins. (38) It is noteworthy that this chemistry was conducted completely without the use of carbohydrate protecting groups;

FIG. 3 shows additional synthetic steps in synthesizing a glycodendrimeric protease. A, a, pentaacetyl-Gal, BF3.Et2O, DCM, 76%; b, MeONa, MeOH, 83%; c, NaSSO2CH3, DMF, 78%; see Ref. (21) B, d, NaSSO2CH3, DMF, 56%; e, 2 equiv. Gal-S-Na+, DMF, 0° C. 55%. C, f, Boc2O, DCM, −78° C., 68%; g, (ClCH2CO)20, DCM, 97%; h, 2 equiv. Gal-S-Na+, DMF, 88%; i, CF3COOH, DCM, 91%; j, thiobutyrolactone, dithiothreitol, NaHCO3, water, EtOH, 69% k, NHS-butyl-MTS, DMF, 81%; l, 1 equiv. Gal-S-Na+, DMF, 48%; m, 119, DMF, 78%; n, CF3COOH, DCM, 94%; o, NHS-butyl-MTS, DMF, 77%; p, NHS-butyl MTS, DMF, 87%; q, 9, DMF, 81%; r, CF3COOH, DCM, 93%; s, NHS-butyl-MTS, DMF, 72%;

FIG. 4 shows mass spectrometric characterization of glycoproteins. ESMS analysis confirmed the formation, with correct mass, of the expected glycoproteins S156C-2a, 2b, 3, 4;

FIG. 5 shows percent aggregation of the pathogen A. naeslundii to S. oralis (after 10 min) after treatnent with glycoprotein enzyme (1.5 μg/mL, 30 min). Most potent inhibition of the binding of A. naeslundii was found following treatment with the biantennary synthetic glycoprotein S1156C-2b (IC50 20 nM) and is >106 more potent than that of small molecule inhibitor lactose (IC50 33 mM);

FIG. 6 shows results from a coaggregation assay used to demonstrate the principle of the invention. Actinomyces naeslunii T14V and Streptococcus oralis 34 were grown in Todd Hewitt broth supplemented with 2% yeast extract and grown at 37° C. in 5% CO₂ and H₂:CO₂:N₂ (10:10:80) respectively, and harvested in mid- to late-exponential phase by centrifugation at 10,000×g at 4° C. for 10 min. Bacteria were washed twice and resuspended to A₅₄₀=1.0 in coaggregation buffer (CB: 1 mM tris(hydroxymethl)aminomethane, 10⁻⁴ M CaCl₂, 10⁻⁴ M MgCl₂, 0.15 M NaCl, pH 7.0).

Cell suspensions of A. naeslundii TI4V were incubated with lactose, modified-proteases (S156C (1-4)), native protease, PMSF+S156C (1-4) and PMSF alone at 37° C. for 1 hr. After incubation an equal volume of S. oralis 34 was added and vortexed for 10 sec. Coaggregation was assessed by turbidity changes at 540 nm over 10 min—decreased turbidity indicating increased aggregation.

This figure displays the concentration dependence of inhibition by the S156C biantennary glycodendrimer, compared to native protease without galactose substitution. 100 mM lactose (digalactoside) results in no loss of turbidity (i.e. results in an absence of aggregation). Adding 2 mM lactose does not block aggregation (i.e., the loss in turbidity is just as extensive as in the control with nothing added). PMSF treatment of the dendrimer results in near-total abrogation of the dendrimer's inhibitory activity since the enzyme has no catalytic activity. This demonstrates that the failure to aggregate is dependent on the proteolytic activity of the glycodendrimer. Note also that 1.5 μg/ml (500 nM) of S156C digal is more effective at blocking aggregation than is 15 μg/ml of the native enzyme and that 4.5 μg/ml S156C-digal is more effective than 50 μg/ml native enzyme. In each case the 10-fold lower concentration of the glycodendrimeric protease yielded less aggregation that the native enzyme at a 10×higher concentration. At 50 μg/ml of S156C-digal (˜1.5 μM) we see almost complete elimination of aggregation, the same level seen in the presence of 100 mM lactose;

FIG. 7 shows a normal addition synthetic scheme for producing two different first-generation Type A (TREN-type)glycodendrimer reagents, and glycodendriproteins produced from these reagents;

FIG. 8 shows a normal addition synthetic scheme for producing a second generation Type A glycodendrimer reagent;

FIG. 9 shows a visualization of reduced aggregation. Samples of the control aggregation reaction and the reaction containing 15 μg/ml S156C-digal were transferred to glass slides for microscopic examination. The photmicrographs shown in FIG. 9 confirm that decreased turbidity indeed correlates with decreased aggregation. A control is pictured on the left. It displays large aggregates of the two bacterial species. The reaction on the right was that in which A. naeslundii was incubated with the biantennary dendrimer (15 μg/ml) before S. oralis was added. It is clear that the dendrimer could significantly reduce the accumulation of bacteria on surfaces that were already colonized with one of the coaggregating partners.

### DETAILED DESCRIPTION OF THE PREFERRED EMBODIMENTS

Unless defined otherwise herein, all technical and scientific terms used herein have the same meaning as commonly understood by one of ordinary skill in the art to which this invention belongs. Singleton, et al., Dictionary of Microbiology and Molecular Biology, 2D Ed., John Wiley and Sons, New York (1994), and Hale & Marham, The Harper Collins Dictionary of Biology, Harper Perennial, N.Y. (1991) provide one of skill with a general dictionary of many of the terms used in this invention. Although any methods and materials similar or equivalent to those described herein can be used in the practice or testing of the present invention, the preferred methods and materials are described. Numeric ranges are inclusive of the numbers defining the range. Unless otherwise indicated, nucleic acids are written left to right in 5′ to 3′ orientation; amino acid sequences are written left to right in amino to carboxy orientation, respectively. Practitioners are particularly directed to Sambrook el al., 1989, and Ausubel FM et al., 1993, for definitions and terms of the art. It is to be understood that this invention is not limited to the particular methodology, protocols, and reagents described, as these may vary.

The headings provided herein are not limitations of the various aspects or embodiments of the invention which can be had by reference to the specification as a whole. Accordingly, the terms defined immediately below are more fully defined by reference to the specification as a whole All publications cited herein are expressly incorporated herein by reference for the purpose of describing and disclosing compositions and methodologies which might be used in connection with the invention.

Definitions

As used herein, a “glycodenrimeric protease” is an enzyme attached via disulfide linkage to a free thiol of a cysteine residue at one or more pre-seleted sites. The glycodendrimeric protease may have a branched or multiply-branched structure that presents a carbohydrate moiety at each branch extremity. Carbohydrate moieties may be selected from the group consisting of glucose, galactose, fucose, mannose, sialic acid, N-Acetylgalactosamine, N-Acetylglucosamine, and glucuronic acid. Useful glycodendrimeric proteases of the invention are shown in FIG. 2. Useful examples can also be found, for example, in WO 02/079394.

Such an enzyme preferentially modifies an adhesin on a microorganism, such as a protein, by binding to the adhesin followed by proteolytic degradation of the adhesin. For example, such an enzyme can catalyze a reaction for modifying a molecule on the microorganism. An enzyme employed in a method or composition of the invention can, for example, catalyze a degradation of the binding site of an adhesin, such as a lectin or another carbohydrate binding site, on the microorganism. Preferably, the enzyme has one or more branched dendric antennae presented.

As used herein, “microorganism” refers to microbes including a eukaryote, a prokaryote or a virus and including, but not limited to, a bacterium (either gram positive, gram negative or gram-variable), a fungus, a virus, a protozoan and other microbes or microscopic organisms.

As used herein, “adhesin molecule” or “adhesin” means a molecule or complex of molecules that is typically expressed on the surface of a microorganism and that mediates adhesion by the microorganism to cells, tissues, extracellular matrix, teeth, a dental prosthesis, a medical device or catheter or another surface. Some adhesin molecules bind to a receptor on the surface of the other cell, tissue or extracellular matrix. Some adhesin molecules adhere to polysaccharides that coat teeth, gums, dental prostheses and the other tissues in the oral cavity. Some adhesin molecules adhere to polysaccharides or other molecules that coat body cavities, and tissues in these cavities, including the middle ear, vagina and the like or to other microorganisms that infect these cavities. Adhesins include carbohydrate binding proteins or sites on the surface of microorganisms. Adhesin molecules can be found in, on or as parts of lectins, proteins, enzymes, such as glucosyltransferases, hydrophobins, outer membrane proteins, flagella, fimbriae, pili, fibrillae and the like.

Microorganisms can adhere to carbohydrate portions of extracellular matrix (glyco)proteins; alternatively, bacteria can attach to protein portions of extracellular matrix molecules via microbial carbohydrates. A prominent example of this phenomenon is the interaction of the hyaluronic acid capsular polysaccharide of Streptococcus pyogenes with CD44 (protein) molecules on human cells (Courtney). Also, many conserved carbohydrate-containing bacterial components such as lipopolysaccharide and peptidoglycan (all displaying PAMPs—pathogen associated molecular patterns) have been found to bind to toll-like-receptors (TLR's) on a variety of mammalian cells (Kirschning). A third example is the colonization of teeth by S. mutans, which is in part mediated by the bacterium's association with tooth-anchored glucosytransferases via glucan bound to its surface.

As used herein, TREN refers to tris(2-aminoethyl)amine.

As used herein, “adhesion by a microorganism” refers to the binding of a microorganism to a cell, tissue, extracellular matrix, a tooth, a dental prosthesis or another surface, including hard surfaces that are cleaned by detergents or cleaners. The surface can be of a body cavity such as the oral cavity, vagina, middle ear or the like.

As used herein, “reduce adhesion by a microorganism” or “reducing adhesion by a microorganism” refers to decreasing the amount of adhesion by the microorganism to a cell, tissue, extracellular matrix, a tooth and/or dental prosthesis or to any other surface onto which microorganisms adhere and colonize. The decrease in adhesion can be observed by employing comparison to a control cell, tissue, extracellular matrix, a tooth and/or dental prosthesis or to a control population. Generally, “reduce” or “reducing” can also be expressed as inhibit or inhibiting, diminish or diminishing, abolish or abolishing and like terms. Reduction in adhesion by a microorganism by an amount that is measurable with statistical significance as less than a control value for adhesion by the microorganism can be expressed as “significantly reduced adhesion by a microorganism”. Significant reduction in adhesion by a microorganism can also be determined by demonstrating a desired biological effect upon treatment of a microorganism with a glycodendrimeric protease, preferably including correlation of this effect with adhesion by the microorganism.

As used herein, “dental prosthesis” refers to a replacement for one or more of a mammal's teeth or another oral structure, including replacement of a single tooth, any type of denture and any type of bridge. A dental prosthesis can be either fixed in the mammal's oral cavity or removable from the mammal's oral cavity. As used herein, “denture” refers to any type of denture including a partial denture, a complete denture, a fixed denture and a removable denture.

As used herein “surface” refers to any surface to which a microorganism can bind or adhere. Surfaces include-cells, tissues or extracellular matrix, among others. Surfaces also include the surface of any catheter, implant, prosthesis or other man made device that resides or is placed in or on a mammal's body or body cavity. Surfaces also include other surfaces to which a microorganism might bind such as a surface of a medical device external to the mammal, but that contacts the mammal or mammalian fluids or tissues, such as a periodontal dialysis apparatus, kidney dialysis apparatus, heart/lung machines and the like. Surfaces also include surfaces in other apparatus or equipment to which microorganisms can adhere, such as in brewing apparatus, fermentation apparatus, effluent treatment apparatus and other reactors and apparatus. Surfaces include hard surfaces that are cleaned by detergents or other cleaners. As used herein, the terms “treating”, “treatment” and “therapy” refer to curative therapy, prophylactic therapy and preventative therapy. Treating, treatment and therapy can reduce or ameliorate the severity or presence of symptoms of a disorder, can reduce or ameliorate the severity or presence of a disorder or can cure the disorder.

As used herein, the term “mammal” refers to any mammal classified as an animal, including humans, cows, horses, dogs and cats. In a preferred embodiment of the invention, the mammal is a human.

As used herein, the term “animal” refers to vertebrate animals including birds, mammals, reptiles, amphibians and the like. Preferred animals include mammals and birds.

As used herein, the term “composition” refers to substances that can be administered to a subject, preferably a mammal, to treat a disorder that may benefit from administering an enzyme, such as a glycodendrimeric protease, to reduce adhesion by one or more microorganisms.

As used herein, the term “oral care composition” refers to a composition suitable for administration to the oral cavity of a subject, preferably a mammal, to treat a disorder of or in the oral cavity that may benefit from administering an enzyme, such as a glycodendrimeric protease, to reduce adhesion by one or more microorganisms.

As used herein, the term “effective amount” refers to an amount of a glycodendrimeric protease sufficient to reduce or inhibit adhesion by a microorganism to a cell, tissue, extracellular matrix, a tooth, dental prosthesis or another surface, including hard surfaces that are cleaned by detergents or cleaners.

As used herein, the term “glycodendrimer” refers to chemically synthesized branching structures bearing carbohydrates on their branch tips. Glycodendrimers can be found, for example, in WO 02/079394.

As used herein, the terms “highly branched molecules” or “dendrimers” or “well-defined branching dendrimers” are synonymous and refer to structures of the invention as herein described. Highly branched molecules were first were synthesized by Vögtle in 1978 (Buhleier et al., Synthesis, 155-158 (1978)). The attachment of identical building blocks that contain branching sites to a central core may be achieved with a high degree of homogeneity and control. Each branch contains a functional group which, after chemical alteration, may be connected to yet another branching building block. In this manner, layer after layer of branching rapidly generates highly-functionalized molecules.

For instance, multiple glycosylation, including multiple mannose-containing chemically modified mutant proteins, and varied sugar moieties, can be created. The dendrimer reagent structures would include methanethiosulfonates with simple branching such as:

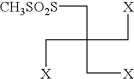

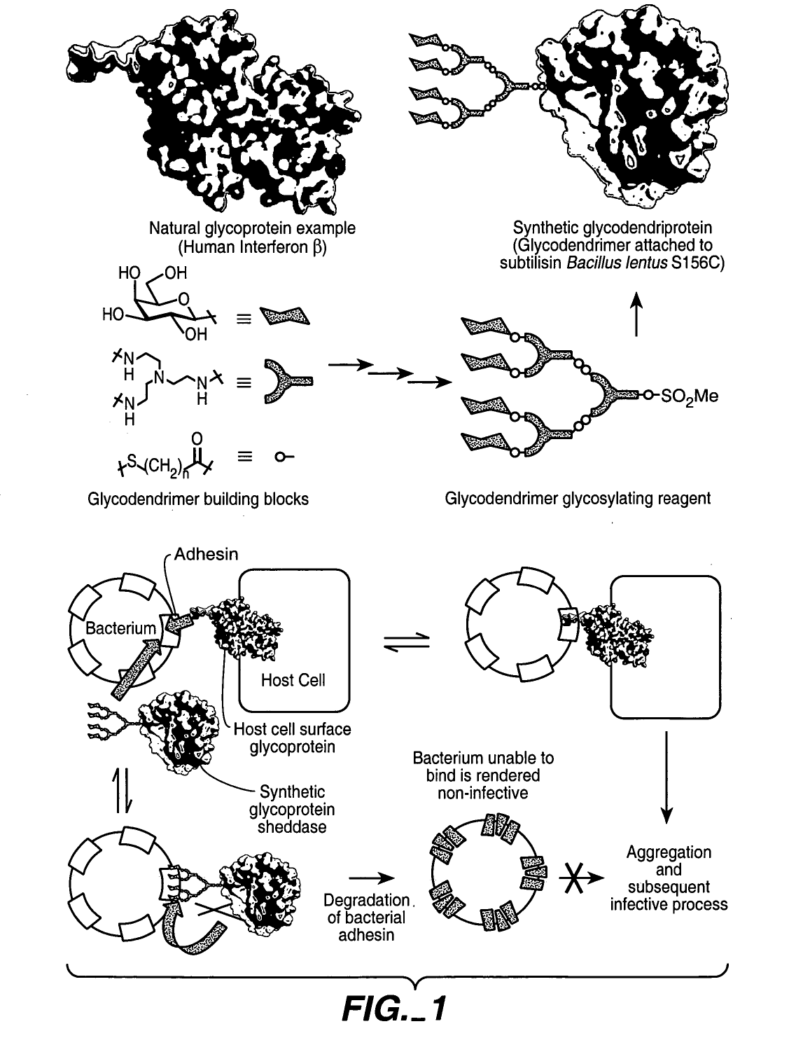

derived from pentaerythritol (i.e., “Penta-E”), to very complex branched dendrimer reagents (e.g., see FIG. 1). In particular, a first generation glycodendrimer reagent is synthesized as shown in FIG. 7. This approach can be extended to cover larger dendrimers. More specifically, by leaving one “arm” of the glycodendrimer free for conversion to a methanethiosulfonate, the remaining arms can be further branched to synthesize highly-functionalized glycodendrimer reagents as shown in FIG. 8.

Glycodendrimeric proteases are employed to degrade the adhesin, and the enzyme can be modified. Preferred enzymatic modifications employ glycodendrimeric proteases having an appropriate branched structure to optimally present an isomeric form of the carbohydrate moieties selectively recognized by a particular adhesin or lectin. It is believed that the glycodendrimeric protease can act to inhibit cellular adhesion be selectively degrading the adhesion recognition site for attachment to specific cell surface glycoproteins on the host cell surface. Blocking this adhesion prevents a series of events including a tight binding event that can lead to infection and disease. In another scenario, tight binding can lead to the synthesis of components that will facilitate forming supportive biofilms. It is believed that by blocking the adhesion step by eliminating the adhesin recognition sites that the glycodendrimer functions as an anti-infective agent. It is believed that this glycodendrimer anti-infective agent could be effective alone or in combination with other antibiotics to enhance the antibiotic's function by prevention of attachment and/or the formation of biofilms which are believed to render the bacteria more resistant to antibiotic microbials. The selectivity of the glycodendrimer proteases may also have additional beneficial properties by targeting harmful bacterial while leaving innocuous and benevolent bacterial unaffected. The growth of innocuous bacterial is in itself potentially beneficial by virtue that these bacterial will compete for available nutrients with harmful bacteria thereby further limiting their growth.

Methods and Compositions

The present invention includes methods and compositions employing an enzyme, such as a glycodendrmeric protease, for reducing adhesion by a microorganism, preferably without killing or halting the growth of the microorganism. The methods and compositions of the invention can reduce or inhibit binding or adhesion of a microorganism to a cell, tissue or other surface. Further, the glycodendrimer, comprised of the same carbohydrate recognized by the bacterial adhesin, specifically directs the enzyme to bind at the site which would ordinarily be used by the bacterium to bind the target receptor, such as a carbohydrate, and therefore can be effective in much lower concentrations and have many fewer global effects on the bacterial cell and/or body site in which it is employed. The methods of the invention include administering effective amounts of the enzyme, for reducing or preventing adhesion by a microorganism, for example, at the site of an infection by a microorganism in an animal's body, including a body cavity, a dental prosthesis, a tissue, a site of catheterization or the like. The compositions of the invention include effective amounts of a glycodendrimeric protease, in a carrier suitable for maintaining this enzyme in a form active for reducing adhesion by a microorganism. In one embodiment, the composition includes a pharmaceutical composition, suitable for therapeutic administration to an animal. The compositions of the invention also include oral care compositions. The compositions of the invention also include compositions suitable for applying an enzyme such as a glycodendrimeric protease to a surface of a prosthesis, medical device (e.g. a catheter), a polymeric surface, a metal surface, or the like that can be cleaned with a cleaner or disinfectant.

The enzyme employed in the methods or compositions of the invention preferentially modifies an adhesin, such as a carbohydrate binding site, on the microorganism. Such an enzyme can catalyze a reaction for modifying an adhesin or other molecule on the microorganism or in the binding site of a lectin or another carbohydrate binding site on the microorganism. Adhesion by a microorganism can occur through a variety of mechanisms to a variety of substrata. For example, microorganisms that inhabit an animal's oral cavity can adhere to polysaccharides that coat teeth, gums, tongue, throat, cheeks, a dental prosthesis and the other tissues in the oral cavity. Microorganisms also can adhere to carbohydrates on cells, tissues and extracellular matrix in or on the animal's body or in or on one of the animal's body cavities. The cells can include other microorganisms. Such microorganism to microorganism binding can be referred to as coaggregation. Microorganisms that coaggregate include microorganisms that are early and late colonizers of freshly cleaned teeth. Microorganisms can also adhere (less frequently) to proteins on cells, tissues, and extracellular matrix via microbial carbohydrate residues. In this case, the enzyme employed in the methods or compositions of the invention preferentially modifies a carnohydrate-binding residue on the host cell.

Microorganisms frequently employ adhesins, including carbohydrate binding structures such as lectins and glucosyltransferases. Microorganisms that employ adhesin molecules include bacteria, such as Actinobacillus actinomycetemcomitans, Actinomyces israelii, A. naeslundii and A. viscosus, Capnocytophaga ochracea, Eikenella corrodens, Escherichia coli, Fusobacterium nucleatum, Haemophilus influenzae, Porphyromonas gingivalis, Prevotella intermedia, Proteus mirabilis, Proteus vulgaris, Pseudomonas aeruginosa, P. loeschei, Streptococcus gordonii, S. mutans, S. oralis, S. sanguis, various group A streptococci, various invasive and antibiotic resistant staphylococci, and Treponema denticola; viruses such as influenza virus; yeasts, such as Candida albicans; and protozoans, such as Entamoeba histolytica. Adhesin molecules of several M5, M6 and M24 positive strains of streptococci have been studied (Dale J. B. et al., Vaccine 14-944-948 (1996); Courtney et al., FEMS Microbiol. Letters 151:65-70 (1997)). P. aeruginosa makes a good model for study as its adhesion can depend on two lectins, PA-1 and PA-2. Furthermore, the bacterium will form biofilms on a variety of surfaces, ranging from glass and steel to human lungs.

Adhesion by a microorganism can be determined by employing a variety of techniques known to those of skill in the art. These techniques include determining coaggregation of the microorganism of interest with a cell, such as through turbidimetry, determining aggregation of microorganisms with a polysaccharide, such as formation of an aggregate in solution or a pellicle, determining binding of the microorganism to a mammalian cell, monitoring hemagglutination and determining binding of a microorganism to extracellular matrix.

Typical assays for aggregation or coaggregation including microorganisms can be done in a suitable buffer and can involve visual end-point estimates or kinetic measurements, such as those employing a platelet aggregometer. Visual end-points can be determined by methods known to those of skill in the art, such as those described by Kolenbrander (Kolenbrander, P. E., Meth. Enzymol. 253:385-396 (1995)). A more quantitative method for measuring coaggregation involves mixing the two coaggregating partners together in a test tube and observing the decrease in the optical density of the mixture over time. As aggregates form they settle to the bottom of the tube and the rate and extent of settling can be measured by the decrease in optical density. The decrease is measured continuously in a spectrophotometer and plotted on a graph of optical density vs. time.

Suitable reaction pairs of microorganisms for measuring co-aggregation include:

| | | Control Reac- |
|---|---|---|
| Contains adhesin | | tion can Employ |
| molecule: | Contains receptor: | Protection by: |
| A. naeslundii T14V | S. oralis 34 | Lactose |
| Capnocytophaga | A. viscosus | Rhamnose |
| ochracea | | |
| F. nucleatum | A. israelii | Lactose |
| F. nucleatum | A. actinomycetemcomitans | Unknown |
| Prevotella intermedia 27 | A. naeslundii | None |
| Prevotella loeschei | S. oralis | Lactose |
| S. gordonii | A. naeslundii | None |
| S. sanguis | Porphyromonas gingivalis | None |
| | W50 | |

Typically, in these pairs of microorganisms, the adhesin molecule is inactivated by heat and/or pronase, indicating that it is a protein, but the receptor is resistant to heat and/or pronase, meaning it is a non-protein, typically, a carbohydrate. These microbes represent early and late colonizers, Gram-positive and Gram-negative, those susceptible to protection by carbohydrates and those resistant to effects of carbohydrates. Nearly always one of the bacterial partners uses a protein to bind to a carbohydrate on its partner bacterium, a type of reaction particularly amenable the current invention. Additional coaggregating pairs have been employed in studies reported, as described in the Figures and Examples provided herein.

Adhesion of a microorganism to a cell from an animal tissue can be determined by any of a variety of methods known to those of skill in the art. Such methods include, for example, assaying E. coli strains possessing Pap-type fimbriae for adhesion to their substratum, di-galactose, by mixing them with suspensions of latex beads conjugated with the disaccharide (EY Laboratories) according to the procedure of Garcia et al. (Garcia E. et al., Curr. Microbiol. 17:333-337(1988)). For adhesion of the group A streptococci, human laryngeal cells (HEp-2 from ATCC) or sloughed buccal epithelial cells can be used as substrata. For such assays, bacteria can be tested at a variety of densities, starting, for example, at a high of 10⁹/ml with dilutions down to about 10⁷/ml or lower. These ranges can be used to generate a binding isotherm as described in Chapter 2 of Ofek and Doyle, 1994 supra. The adhesion reaction mixture can be incubated then aspirated and washed with medium to remove non-adherent or adventitiously bound cells. The data obtained may yield, for example, regular binding isotherms, Langmuir plots, Scatchard plots and/or analysis of “cooperative” adhesion (Ofek and Doyle, supra). For example, if the glycodedrimeric protease of the present invention abolish a positive slope of a Scatchard plot of adhesion results, it could be said the enzyme is preventing positive cooperativity.

C. albicans is a fungal microorganism that can infect denture wearers, headneck irradiated patients, Sjögren's patients, AIDS patients, and other immunocompromised subjects. Adhesion of C. albicans to various substrata can be determined employing the general procedures of Hazen and Glee (Hazen, K. C. and Glee, P. M., Meth. Enzymol. 253:414-424 (1995)) and of Segal and Sandovsky-Losica (Segal, E. and Sandovsky-Losica, H., Meth. Enzymol. 253:439-452 (1995)). C. albicans for adhesion studies can be obtained from exponential (yeast phase) cultures in YE (yeast extract).

A variety of microorganisms, such as oral bacteria, can adhere to many substrata including various extracellular matrix proteins. Adhesion to extracellular matrix proteins can be measured by a variety of methods known to those of skill in the art. For several oral bacteria, fibronectin is a receptor for their adhesin molecules. For others, collagen serves as a receptor. Other receptors are also known. (Ljungh, A. and Wadstrom, T. Meth. Enzymol. 253:501-573 (1995)). Bacteria known to adhere to collagen include Actinomyces viscosus, Porphyromonas gingivalis, and Prevotella intermedia. (see, for example, Liu, T., R. J. Gibbons, D. I. Hay, and Z. Skobe, Oral Microbiol Immunol. 6:1-5 (1991); Naito, Y., and R. J. Gibbons, J. Dent. Res. 67:1075-1080; Grenier, D., include S. sanguis, S. pyogenes M5⁺ protein and Treponema denticola (reviewed in Ofek & Doyle, Bacterial Adhesion to Cells and Tissues. Chapman and Hall, New York. 1994). Adhesion to collagen can be studied by the method described by Grenier 1996 supra; and experiments with fibronectin can be patterned after those with collagen. Microorganisms can adhere to carbohydrate portions of extracellular matrix (glyco)proteins; alternatively, bacteria can attach to protein portions of extracellular matrix molecules via microbial carbohydrates and/or proteins.

The microorganisms employed in studies of adhesion can be produced and isolated by any of a variety of methods known to those of skill in the art. For example, microorganisms can be purchased, obtained from clinical isolates, or prepared in other ways. Protozoa such as E. histolytica can be grown axenically as described by Petri and Schnaar (Petri, W. A. Jr. and Schnaar, R. L., Meth. Enzymol. 253:98-104 (1995)). The trophozoites can be harvested and washed as described. Adhesion studies can employ trophozoite membranes and hemagglutination to assay for the lectin. Viruses, such as influenza A virus, can be cultured, harvested, and handled according to procedures well known in the art.

Administering an effective amount of an enzyme, such as a glycodendrimeric protease, to animal tissues, cells, extracellular matrix, teeth and/or dental prosthesis preferably results in a decrease in adhesion by one or more microorganisms sufficient to ameliorate detrimental effects or disease resulting from such adhesion. Effective administration or use of the enzyme, such as a glycodendrimeric protease, in this manner is typically evidenced by prevention or inhibition of infection, reduction or moderation of symptoms of an infection, reduction of adhesion, and the like. Absence or reduction of infection and moderation of symptoms can be determined by common clinical or laboratory methods. Reduction of adhesion can be determined by plate counts, microscopy, aggregometry, turbidimetry, isotopic labeling and other methods standard in the art.

An enzyme, such as a glycodendrimeric protease, that decreases adhesion can be useful in one or more of a variety of applications including: fighting biofouling, for example in peritoneal dialysis; reducing dental caries; treating symptoms of infection by reducing adhesion of E. coli in an animal's gut; treating infection by reducing adhesion by one or more protozoa, such as Entamoeba; treating ulcers, for example by reducing adhesion of Helicobacter; treating viral infections by reducing adhesion of viruses, such as influenza virus; serving as a birth control agent, since sperm and egg interact via similar lectin-mediated interactions as described for microorganisms; reducing contamination of eggs and/or other poultry products by serving as a chicken feed supplement for reducing levels in the bird of salmonellae; treating infection of periodontal tissue, eye, ear, or throat, such as by reducing adhesion by Haemophilus, Streptococcus, or Candida; as a component of eye or ear drops, of a gargle (e.g. for sore throat), of a gels in a periodontal disease packing; killing mosquito larvae when cloned into Bt; as a probiotics (e.g. by cloning glycodrimeric protease into Lactobacillus); or for fighting skin infections (impetigo) caused by Staphylococcus or Streptococcus or Vibrio cholerae infections (caused by Vibrio toxin binding to CHO) and other bacterial diseases which rely on toxin binding to host tissues, such as whooping cough and diphtheria.

The methods and compositions of the present invention can be employed to treat urinary tract infections. Such infections are responsible for 9.6 million physician visits per year. The vast majority of these are caused by E. coli possessing various fimbrial structures. Nearly all E. coli strains express type 1 fimbriae, and certain allelic variants of the fimbriae are associated with the ability to colonize the lower urinary tract, utilizing mannose on host cells for attachment. P-fimbriated E. coli use digalactose structures for attachment and are strongly associated with upper urinary tract (i.e., kidney) infections.

The methods and compositions of the present invention can be employed to treat infections at sites of catheters and/or cannulas. Organisms such as Proteus mirabilis, Proteus vulgaris and Pseudomonas aeruginosa frequently colonize catheters, resulting in catheter removal and/or infection in the subject. Adhesion can lead to encrustation because the ammonia from urease will increase pH enough to precipitate struvite (Mg—NH₄-phosphate) and hydroxylapatite (Ca phosphate). It may be that a glycodendrimeric protease can inhibit urease and/or adhesion. Or it may be that the enzymes reduce adhesion but not have any effects on urease. In either case, it is desired to reduce encrustation and prolong the life of the catheter. A model provided in some detail by Tunney et al. (1999, Biofilm and biofilm-related encrustation of urinary tract devices. Meth. Enzymol. 310:558-566) can be employed to demonstrate the effectiveness of an enzyme such as a glycodendrimeric protease against such adhesion related encrustation. Catheters and like instruments can be by methods and in formulations known to those of skill in the art for inclusion in cleaning and/or sanitizing compositions. In certain circumstances such enzymes can be employed during the process or treatment effected by the device or apparatus to reduce adhesion of microorganisms.

Pharmaceutical Compositions

In one embodiment of the invention, there are provided pharmaceutical compositions including an enzyme, such as a glycodendrimeric protease. An enzyme, such as a glycodendrimeric protease, can be used in such pharmaceutical compositions, for example, for the treatment of microorganismal pathologies. It is contemplated that the pharmaceutical compositions of the present invention can be used to treat infections by one or more microorganisms that rely upon an adhesin molecule with a binding site.

The pharmaceutical compositions of the present invention preferably contain an effective amount of an enzyme, such as a glycodendrimeric protease, to reduce adhesion by a microorganism. Optionally, the pharmaceutical composition may include agent(s) that stabilize or augment the activity of a glycodendrimeric protease. Such agents include, but are not limited to, starch, gelatin, carrageenan, glycols and other agents used to compound pharmaceuticals.

The pharmaceutical compositions of the present invention include an enzyme, such as a glycodendrimeric protease, in a pharmaceutically acceptable carrier. Pharmaceutically acceptable carriers are known to those skilled in the art and include materials useful for the purpose of administering a medicament, which are preferably non-toxic, and can be solid, liquid, or gaseous materials, which are otherwise inert and medically acceptable and are compatible with the enzyme, such as a glycodendrimeric protease, and any other active ingredient that is present.

Water, saline, aqueous dextrose, and glycols are preferred liquid carriers, particularly when isotonic, for injectable solutions. The carrier can be selected from various oils, including those of petroleum, mammal, vegetable or synthetic origin, for example, peanut oil, soybean oil, mineral oil, and sesame oil. Suitable pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, and ethanol. The compositions can be subjected to conventional pharmaceutical expedients, such as sterilization, and can contain conventional pharmaceutical additives, such as preservatives, stabilizing agents, wetting, or emulsifying agents, aerosolizing agents, salts for adjusting osmotic pressure, or buffers. Suitable pharmaceutical carriers and their formulations are described in Martin, “Remington's Pharmaceutical Sciences,” 15th Ed.; Mack Publishing Co., Easton (1975); see, e.g., pp. 1405-1412 and pp. 1461-1487. Such compositions will, in general, contain an effective amount of an enzyme, such as a glycodendrimeric protease, to reduce adhesion by a microorganism, together with a suitable amount of carrier so as to prepare the proper dosage form for proper administration to the animal.

The pharmaceutical compositions of the invention can be administered by various routes, including orally, used as a suppository or pessary; applied topically as an ointment, cream, aerosol, powder; or given as eye or nose drops, etc., depending on whether the preparation is used to treat internal or external infections by one or more microorganisms. The compositions can contain 0.1%-99% of the enzyme, such as a glycodendrimeric protease. Preferably, the composition includes about 0.1 wt-% to about 1.0 wt-% of an enzyme, such as a glycodendrimeric protease. The enzymes are usually soluble in pharmaceutical preparations.

For oral administration, fine powders or granules can contain diluting, dispersing and/or surface active agents, and can be presented in a draught, in water or in a syrup; in capsules or sacnets in the dry state or in a non-aqueous solution or suspension, wherein suspending agents can be included; in tablets or enteric coated pills, wherein binders and lubricants can be included; or in a suspension in water or a syrup. In some cases, the enzyme(s) may be formulated to form aerosols. Where desirable or necessary, flavoring, preserving, suspending, thickening, or emulsifying agents can be included. Tablets and granules are preferred, and these can be coated. A preferred formulation for oral administration includes agents that maintain the activity of an enzyme, such as a glycodendrimeric protease, in the stomach and intestines. Such agents include buffers and “slow release” components.

For buccal administration, the compositions can take the form of tablets or lozenges formulated in a conventional manner.

Alternatively, for infections of the skin or other external tissues the compositions are preferably applied to the infected part of the body of the animal as a topical ointment, cream or spray. The enzyme, such as a glycodendrimeric protease, can be presented in an ointment, for instance with a water-soluble ointment base, or in a cream, for instance with an oil in water cream base. Carriers for topical or gel-based forms of include polysaccharides such as methylcellulose, polyvinylpyrrolidone, polyacrylates, polyoxyethylene-polyoxypropylene-block polymers, polyethylene glycol, and wood wax alcohols. For topical administration, an enzyme, such as a glycodendrimeric protease, can be present in the pharmaceutical composition in a concentration of from about 0.01 to 10%, preferably 0.1 to 1.0% w/v. For topical administration, the daily dosage as employed for adult human treatment will range from 0.1 mg to 1000 mg, preferably 0.5 mg to 10 mg. However, it will be appreciated that extensive skin infections can require the use of higher doses.

For all administrations, conventional depot forms are suitably used. Such forms include, for example, microcapsules, nano-capsules, liposomes, plasters, inhalation forms, nose sprays, sublingual tablets, and sustained-release preparations. The enzyme, such as a glycodendrimeric protease, will typically be formulated in such carriers at a concentration of about 0.1 mg/ml to 100 mg/ml. An enzyme, such as a glycodendrimeric protease, can also be administered in the form of a sustained-release preparation. Suitable examples of sustained-release preparations include semipermeable matrices of solid hydrophobic polymers containing the protein, which matrices are in the form of shaped articles, e.g., films, or microcapsules. Examples of sustained-release matrices that are well known in the art include polyesters, hydrogels, polylactides, copolymers of L-glutamic acid and gamma ethyl-L-glutamate, nondegradable ethylene-vinyl acetate, degradable lactic acid-glycolic acid copolymers, and poly-D-(−)-3-hydroxybutyric acid.

An enzyme, such as a glycodendrimeric protease, can also be administered employing a composition suitable for gene therapy. For in vivo delivery of a nucleic acid (optionally contained in a vector) into an animal's cells, the nucleic acid is injected directly into the animal, usually at the sites where the polypeptide is required. Known in vivo nucleic acid transfer techniques include transfection with viral or non-viral vectors (such as adenovirus, lentivirus, Herpes simplex I virus, or adeno-associated virus (AAV)) and lipid-based systems (useful lipids for lipid-mediated transfer of the gene are, for example, DOTMA, DOPE, and DC-Chol; see, e.g., Tonkinson et al., Cancer Investigation, 14(1): 54-65 (1996)). A viral vector typically includes at least one element that controls gene expression, an element that acts as a translation initiation sequence, a packaging signal, long terminal repeats (LTRs) or portions thereof, and positive and negative strand primer binding sites appropriate to the virus used (if these are not already present in the viral vector). In addition, such vector typically includes a signal sequence for secretion of an enzyme, such as a glycodendrimeric protease, from a host cell in which it is produced.

Oral Care Compositions

The invention further provides oral care compositions including an enzyme, such as a glycodendrimeric protease. An enzyme, such as a glycodendrimeric protease, can be used in such oral care compositions, for example, for the treatment of pathologies in which a microorganism infects the oral cavity. It contemplates that the oral care compositions of the present invention can be used to treat any infection by one or more microorganisms that rely upon an adhesin molecule.

The oral care compositions of the present invention preferably contain an effective amount of an enzyme, such as a glycodendrimeric protease, to reduce adhesion by a microorganism to cells or tissue of the oral cavity or to a dental prosthesis (e.g. a denture). The enzyme, such as a glycodendrimeric protease, is preferably resistant to pasteurization, stable in compounding agents and amenable to formulation as a solid, liquid or aerosol. Optionally, the oral care composition may include agent(s) that stabilize or augment the activity of the enzyme, such as a glycodendrimeric protease. Such agents include trace metals, such as copper ions, and oxygen generating compounds, such as hydrogen peroxide.

Oral care compositions including an enzyme, such as a glycodendrimeric protease, can be used, for instance, for maintaining and/or improving oral hygiene in the oral cavity of mammals, and/or preventing or treating dental diseases in mammals. The present oral care compositions can also be used for reducing adhesion by one or more microorganisms to a dental prosthesis. For example, a denture can be cleaned with an enzyme, such as a glycodendrimeric protease, containing oral care composition either in the wearer's oral cavity or removed from the wearer's oral cavity. Oral care compositions of the invention include but are not limited to toothpaste, a dental cream, gel or tooth powder, a mouth wash or rinse, a denture cleaning agent (e.g. a cream or a soak), a chewing gum, a lozenge, and a candy. The oral care composition can be in the form of a solid, a semi-solid (e.g. a gel, a paste, or a viscid liquid), a liquid, or an aerosol.

Various ingredients that may be included in a tooth paste or gel and a mouth wash or rinse are well known in the art. In addition to an enzyme, such as a glycodendrimeric protease, a toothpaste or gel of the present invention will typically include one or more abrasives or polishing materials, foaming agents, flavoring agents, humectants, binders, thickeners, sweetening agents, or water. An enzyme, such as a glycodendrimeric protease, containing mouth wash or rinse will typically also include a water/alcohol solution and one or more flavors, humectants, sweeteners, foaming agents, and colorants.

Suitable, known abrasives or polishing materials include alumina and hydrates thereof (e.g. alpha alumina trihydrate), magnesium trisilicate, magnesium carbonate, sodium bicarbonate, kaolin, aluminosilicates (e.g. aluminum silicate), calcium carbonate, zirconium silicate, powdered plastics (e.g. powdered polyvinyl chloride, polyamide, or various resins) xerogels, hydrogels, aerogels, calcium pyrophosphate, water-insoluble alkali metaphosphates, dicalcium phosphate and/or its dihydrate, dicalcium orthophosphate, tricalcium phosphate, particulate hydroxylapatite, and mixtures of these abrasives or polishing materials. Typically, the abrasive or polishing material can be present in from 0 to about 75% by weight, preferably from 1% to about 65%, more preferably, for toothpastes or gels, about 10% to about 55% by weight of the toothpaste or gel.

Suitable, known humectants, which are typically employed to prevent loss of water from a toothpaste or gel, or other composition, include glycerol, polyol, sorbitol, polyethylene glycols (PEG), propylene glycol, 1,3-propanediol, 1,4-butane-diol, hydrogenated partially hydrolyzed polysaccharides, and mixtures of these humectants. In a toothpaste or gel, humectants are typically at about 0% to about 75%, preferably about 5 to about 55% by weight of the composition.

Suitable, known thickeners and binders, which maintain stability of an oral care composition include silica, starch, tragacanth gum, xanthan gum, extracts of Irish moss, alginates, pectin, certain cellulose derivatives (e.g. hydroxyethyl cellulose, carboxymethyl cellulose, or hydroxy-propyl cellulose), polyacrylic acid and its salts, and polyvinyl-pyrrolidone. Typically, a toothpaste or gel includes about 0.1% to about 20% by weight of one or more thickeners and about 0.01% to about 10% by weight of one or more binders.

A suitable foaming agent or surfactant in such oral care compositions will typically not significantly decrease the activity of an enzyme, such as a glycodendrimeric protease, present in the composition. Such foaming agents or surfactants can be selected from anionic, cationic, non-ionic, and amphoteric and/or zwitterionic surfactants. These can include fatty alcohol sulphates, salts of sulphonated mono-glycerides or fatty acids having 10 to 20 carbon atoms, fatty acidalbumin condensation compositions, salts of fatty acids amides, taurines, and/or salts of fatty acid esters of isothionic acid. The foaming agent or surfactant can be at levels in the composition from about 0% to about 15%, preferably from about 0.1% to about 10%, more preferably from 0.25 to 7% by weight.

Suitable, known sweeteners include artificial sweeteners such as saccharin and aspartame. Suitable, known flavors include spearmint and peppermint. Such flavors or sweeteners are typically present at levels from about 0.01% to about 5% by weight, or from about 0.1% to about 5%.

The oral care compositions of the invention can also include one or more added antibacterials, anti-calculus agents, anti-plaque agents, compounds which can be used as fluoride source, dyes/colorants, preservatives, vitamins, pH-adjusting agents, anti-caries agents, or desensitizing agents.

An oral care composition including an enzyme, such as a glycodendrimeric protease, can be applied to the oral cavity of a mammal employing any of numerous methods known in the art for administering oral care compositions. For example, the oral care composition can be applied as any commonly applied toothpaste or mouthwash. The oral care composition can be introduced into the oral cavity, applied to an oral tissue, such as teeth and/or gums, removed from the oral cavity (e.g. by rinsing), and the oral cavity can be rinsed. Alternatively, the oral care composition can be applied to the periodontal pocket as a semi-solid or as a solid implant. A gel, paste, or viscid liquid can be applied with, for example, a toothbrush, a swab, a finger, a syringe, or a dentist's tool. In yet another embodiment, the oral care composition can used to soak a denture.

Yet another embodiment of the composition of the invention includes a composition suitable for reducing or inhibiting adhesion or binding of microorganisms to hard surfaces, including dental prostheses, medical devices, implants, counters, porcelain or plastic fixtures, instruments, and the like.

Articles of Manufacture

The invention further provides articles of manufacture. An article of manufacture such as a kit containing an enzyme, such as a glycodendrimeric protease, useful for reducing adhesion by a microorganism, or for the treatment of the disorders described herein, includes at least a container and a label. Suitable containers include, for example, bottles, vials, syringes, and test tubes. The containers may be formed from a variety of materials such as glass or plastic. The container holds a composition that is effective for treating the condition and may have a sterile access port. The active agent in the composition is the enzyme, such as a glycodendrimeric protease. The label on, or associated with, the container indicates that the composition is used for treating the condition of choice. The article of manufacture may further include a second container including a pharmaceutically-acceptable buffer, such as phosphatebuffered saline, Ringer's solution, and dextrose solution. It may further include other materials desirable from a commercial and user standpoint, including other buffers, diluents, filters, needles, syringes, and package inserts with instructions for use. The article of manufacture may also include a second or third container with another active agent as described above.

The present invention may be better understood with reference to the following examples. These examples are intended to be representative of specific embodiments of the invention, and are not intended as limiting the scope of the invention.

### EXAMPLE 1

Competitive Enzyme-Linked Lectin Assays (ELLA).

All incubations were carried out at 37° C. Microtiter plates were coated overnight with peanut agglutinin (PNA) solution (100 ∝L of a 0.1 mg/mL solution in phosphate buffered saline pH 7.2 (PBS)). Excess protein was removed by washing (3·200 ″L of PBS containing 0.05% v/v Tween (PBST)). Coated wells were blocked with bovine serum albumin solution

(150 ∝L of a 1% w/v solution in PBS (PBST)) for 1 h. During this period, serial (×2) dilutions of methyl β-D-galactopyranoside were incubated with glycodendriprotease (at

a dilution of which had been determined to give A₄₁₀ of 0.8-1.0 after 20 min). These solutions were then transferred to the blocked plate for an additional 1 h incubation. These plates were subsequently washed (2·PBST, 1·PBS) prior to development with suc-AAPF-pNA (50 ∝L of a 10 mM solution in 0.1M Tris, pH 8.6). The absorbance at 410 nm was monitored after a 20 min period. Binding constants are based on A₄₁₀ in

the

absence of methyl β-D-galactopyranoside and referenced to the inhibitory potential of an methyl ζ-D-galactopyranoside determined for each plate and assuming a K_{D}=5.34

10⁻⁴. (G. B. Reddy, V. R. Srinivas, N. Ahmad, A. Surolia, J. Biol. Chem. 274, 4500-4503 (1999)) All assays were carried out in duplicate.

### EXAMPLE 2

To assess the ability of these Gal-presenting synthetic glycoproteins we used an enzyme-linked lectin assay (ELLA)(7)(35) that utilized surface-immobilized lectin to mimic the display of surface lectins on bacterial surfaces and the inherent peptidase enzyme activity of SBL as a readout. These revealed a steady increase in affinity for the model Gal-binding lectin peanut agglutinin (PNA) with increasing numbers of Gal-antennae (K_{D} for S156C-1, 2a, 2b, 3, 4˜1.1 [symbol] 10-3, 1.5 [symbol] 10-3, 1.8□10-3, 3.4 [symbol] 10-4, 1.4 [symbol] 10-7 M, respectively). Once such Gal-specfic lectin binding had been established, we evaluated the ability of these synthetic glycoproteins to inhibit the function of pathogens that depend on Gal-binding.

Peanut Agglutinin Surface Binding Assay

| Glycodendrimeric protease | K_{D} |
|---|---|
| S156C-1‡ | 1.1* 10⁻³ M |
| S156C-2a | 1.5* 10⁻³ M |
| S156C-2b | 1.8* 10⁻³ M |
| S156C-3 | 3.4* 10⁻⁴ M |
| S156C-4 | 1.4* 10⁻⁷ M† |

‡Designations correspond to structures shown in FIG. 1.
†Determined by linear extrapolation using various concentration of dendrineric protease and methyl β-D-galactopyranoside.

It can be seen the the affinity of binding to peanut agglutinin follows the number of saccharide moieties attach as sugar presenting antennae following what has been previously observed for surface based glycoprotein recognition by different adhesions.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

We claim:

1. A glycodendrimeric protease, the glycodendrimeric protease having a branched or multiply-branched structure that presents a carbohydrate moeity at each branch extremity, wherein the glycodendrimer protease inhibits adhesion of one or more microorganisms to a surface.

2. The glycodendrimeric protease of claim 1, wherein the carbohydrate moeity is at least one selected from the group consisting of glucose, galactose, fucose, mannose, sialic acid, N-Acetylgalactosamine, N-Acetylglucosamine, and glucuronic acid.

3. The glycodendrimeric protease of claim 1, wherein the surface is selected from the group consisting of cells, tissues, extracellular matrix, teeth and prostheses.

4. The glycodendrimeric protease of claim 1, wherein the microorganism comprises a prokaryote, a eukaryote, a virus or a combination thereof.

5. The glycodendrimeric protease of claim 4, wherein the prokaryote comprises a gram-positive bacterium, a gram-negative bacterium or gram-variable.

6. The glycodendrimeric protease of claim 4, wherein the prokaryote comprises an Actinomyces or E. coli.

7. The glycodendrimeric protease of claim 1, wherein the glycodendrimeric inhibits adhesion by binding to a lectin.

8. A composition comprising a glycodendrimeric protease, the glycodendrimeric protease having a branched or multiply-branched structure that presents a carbohydrate moeity at each branch extremity, wherein the glycodendrimeric protease inhibits adhesion of one or more microorganisms to a surface.

9. The composition of claim 8, wherein the carbohydrate moeity is at least one selected from the group consisting of glucose, galactose, fucose, mannose, sialic acid, N-Acetylgalactosamine, N-Acetylglucosamine, and glucuronic acid.

10. The composition of claim 8, wherein the surface is selected from the group consisting of cells, tissues, extracellular matrix, teeth and prostheses.

11. The composition of claim 8, wherein the microorganism comprises a prokaryote, a eukaryote, a virus or a combination thereof.

12. The composition of claim 8, wherein the prokaryote comprises a gram-positive bacterium, a gram-negative bacterium or gram-variable.

13. The composition of claim 12, wherein the prokaryote comprises an Actinomyces or E. coli.

14. The composition of claim 12, wherein the glycodendrimer inhibits adhesion by binding to a lectin.

15. A method of treating a disease or disorder of a patient in need of such treatment comprising administering to the patient a glycodendrimeric protease or a composition comprising a glycodendrimeric protease, the composition or glycodendrimeric protease having a branched or multiply-branched structure that presents a carbohydrate moeity at each branch extremity, wherein the composition or glycodendrimer protease inhibits adhesion of one or more microorganisms to a surface.

16. The method of claim 15, wherein the surface is selected from the group consisting of cells, tissues, extracellular matrix, teeth and prostheses.

17. The method of claim 15, wherein the microorganism comprises a prokaryote, a eukaryote, a virus, or a combination thereof.

18. The method of claim 18, wherein the patient is an animal or human.

19. The method of claim 15, wherein the patient is human.

20. The method of claim 15, wherein administration is topical.

21. A method for reducing adhesion by a microorganism to mammalian oral tissues or cells or to a dental prosthesis comprising administering a glycodendrimeric protease or composition comprising a glycodendrimeric proteases, the glycodendrimeric protease having a branched or multiply-branched structure that presents a carbohydrate moeity at each branch extremity, wherein the glycodendrimeric protease inhibits adhesion of one or more microorganisms, said administration being to the mammalian oral tissues or cells or to the dental prosthesis.

22. The method of claim 21, wherein the mammalian oral tissues or cells or dental prosthesis are those of a human.

23. The method of claim 22, wherein the administration is topical.

24. An oral care composition comprising an effective amount of a glycodendrimeric protease, the glycodendrimeric protease having a branched or multiply-branched structure that presents a carbohydrate moeity at each branch extremity, wherein the glycodendrimer protease inhibits adhesion of one or more microorganisms to a surface.

25. The composition of claim 24, further comprising one or more substances.

26. The composition of claim 24, wherein said composition is a mouthwash or toothpaste.
